(19) United States
(12) Patent Application Publication
(10) Pub. No.: US 2022/0275050 A1
(43) Pub. Date: Sep. 1, 2022

(54) HIGH YIELD PRODUCTION AND USE OF ENZYMATIC-EXCHANGEABLE PEPTIDE MAJOR HISTOCOMPATIBILITY COMPLEX CLASS I SINGLE CHAIN TRIMER TETRAMER

(71) Applicants: Agency for Science, Technology and Research, Singapore (SG); ImmunoSCAPE Pte. Ltd., Singapore (SG)

(72) Inventors: Evan NEWELL, Singapore (SG); Jack Wee LIM, Singapore (SG); Neeraja KULKARNI, Singapore (SG)

(21) Appl. No.: 17/608,878

(22) PCT Filed: May 6, 2020
(86) PCT No.: PCT/SG2020/050264

§ 371 Date: Nov. 4, 2021

(30) Foreign Application Priority Data
May 6, 2019 (SG) 10201904052S

(51) Int. Cl. C07K 14/74; G01N 33/569; C07K 7/08; C07K 7/06

(52) U.S. Cl. CPC C07K 14/70539; G01N 33/56966; C07K 7/08; C07K 7/06; C07K 2319/20; C07K 2319/50; G01N 2333/70517; G01N 2333/70539

(57) ABSTRACT

The present invention relates to methods to produce an enzymatic-exchangeable peptide Major Histocompatibility Complex Class I (MHC-I) Single Chain Trimer (SCT), or tetramer thereof, constructs encoding same and uses thereof, such as detection or isolation of antigen-specific CD8⁺ T cells. Said SCT comprises, in order from N-terminus to C-terminus, (i) a peptide ligand, (ii) a first linker polypeptide comprising an enzyme-cleavable portion, (i11) a β-2 microglobulin (β2ιτι) polypeptide, (iv) a second linker polypeptide, and (v) a mature MHC-I heavy chain polypeptide. In addition, a method of defining a peptide ligand suitable for successful production of a single fusion protein for peptide exchange is claimed.

### FIELD OF THE INVENTION

The present invention relates to methods to produce an enzymatic-exchangeable peptide Major Histocompatibility Complex Class I (MHC-I) Single Chain Trimer, or tetramer thereof, constructs encoding same and uses thereof, such as the detection of antigen-specific CD8⁺ T cells.

### BACKGROUND OF THE INVENTION

Conditional ligand class I MHC tetramers have been used for the purpose of generating antigen specific peptide-MHC molecules for the detection of antigen-specific CD8⁺ T cells, whereby conditional UV-cleavable class I MHC could be generated for HLA-A2, -A1, -A3, -A11 and -B7 and that such peptide-exchange strategies would permit high-throughput generation of peptide MHC complexes [Bakker A H, et al., Proc Natl Acad Sci USA, 105(10): 3825-30 (2008)]. A limitation of this method is observed UV incompatibility with fluorescent labels, the production of reactive nitroso species and photodamage of MHC-I protein.

Others have independently expressed the soluble heavy chain of the MHC class I and β-2-microglobulin (β2m) in Escherichia coli [Garboczi D N, et al., Proc. Natl Acad. Sci. USA 89(8): 3429-3433 (1992)]. The class I MHC α-chain has the transmembrane region removed, only expressing the extracellular domain and is typically engineered to contain the biotin 15 amino acid recognition tag on the C-terminus. In such preparations, the subunits will be expressed as inclusion bodies within the bacteria. The heavy chain and the light chain, along with the peptide of interest, are refolded, and the enzyme BirA is used to specifically biotinylate the lysine residue within the 15 amino acid recognition sequence. A limitation of this method is often the laborious screening of different refolding conditions using synthetic peptides. It is understood that the closed peptide binding groove in MHC-I protein limits the optimal peptide size, requiring a 10-15 amino acid first linker separating the peptide and the β2m [WO 2015/195531].

There is a need to further improve methods of producing MHC-I single chain trimers bearing and exchangeable peptide within the functional pMHC-I complex recognized by CD8⁺ T cells.

### SUMMARY OF THE INVENTION

The present invention provides methods to overexpress large amounts (mg/L) of secreted pMHC-I protein as single chain trimer (SCT) bearing an exchangeable peptide (>20-mer) within the functional pMHC-I complex recognized by CD8⁺ T cells.

Using a Baculovirus expression vector system, the inventors provide in a preferred embodiment a nucleic acid construct that encodes a MHC-I SCT consisting of (a) a secretion leader upstream of a peptide bearing an enterokinase cleavable linker to the conserved human β2m and a GS-linker to the polymorphic heavy chain bearing a BirA motif for streptavidin tetramerization, and (b) a nucleic acid region bearing unique BamHI and XbaI sites, encoding 56 amino acids (flanking the peptide ligand) for peptide-screening to optimize high yield pMHC-I protein secretion without the need of refolding. In the present invention, the nucleic acid construct not only promotes the secretion of large amounts of pMHC-I protein into the growth media for rapid recovery but also allows peptide reloading upon enterokinase treatment of the pMHC-I SCT protein. The novel constructs are functional as demonstrated by efficient and specific detection of antigen-specific T cells after reloading with relevant peptide antigen(s) and use to stain PBMC from healthy donors with a pre-existing T cell response.

According to a first aspect of the present invention, there is a single chain fusion protein comprising, in order from N-terminus to C-terminus of the fusion protein;

(i) a peptide ligand that stabilizes the MHC-I protein prior to enzyme cleavage;

(ii) a first linker polypeptide comprising an enzyme-cleavable portion;

(iii) a β2m polypeptide;

(iv) a second linker polypeptide; and

(v) a mature MHC-I heavy chain polypeptide.

In some embodiments the peptide ligand is a stabilizing peptide.

In some embodiments the first linker polypeptide is cleavable by an enzyme selected from the group comprising or consisting of Enterokinase, Thrombin, Caspase 1, Caspase 2, Caspase 3, Caspase 4, Caspase 5, Caspase 6, Caspase 7, Caspase 8, Caspase 9, Caspase 10, Factor Xa and Granzyme B.

In some embodiments the first linker polypeptide comprises or consists of about 15, 16, 17, 18, 19, 20 or 21 or more amino acid residues, more preferably about 21 amino acid residues.

In some embodiments the first and second polypeptide linkers comprise at least about 80 percent glycine, alanine and/or serine residues. In some embodiments the first and second polypeptide linkers consist of at least 80 percent glycine, alanine and/or serine residues.

In some embodiments the first linker polypeptide comprises or consists of the amino acid sequence set forth in SEQ ID NO: 2.

In some embodiments the second linker polypeptide comprises or consists of about 15, 16, 17, 18, 19 or 20 or more amino acid residues, more preferably about 20 amino acid residues.

In some embodiments the second linker polypeptide comprises or consists of the amino acid sequence set forth in SEQ ID NO: 3.

In some embodiments the peptide ligand comprises from about 4 to 30 amino acid residues. In some embodiments the peptide ligand consists of 4 to 30 amino acid residues.

In some embodiments the peptide ligand comprises from about 6 to 20 amino acid residues, preferably about 8 to 15 amino acid residues. In some embodiments the peptide ligand consists of 8 to 15 amino acid residues.

In some embodiments the peptide ligand is selected from the group comprising or consisting of the amino acid sequence set forth in SEQ ID NO: 4 (AVFAAASDAK), SEQ ID NO: 5 (AVFDRKSDAK), SEQ ID NO: 6 (KILGRVFFV), SEQ ID NO: 7 (KLAEAIFKL) and SEQ ID NO: 8 (YAETAAFAY). It is understood that the stabilizing peptide may be antigenic or non-antigenic and any antigenicity of the peptide ligand may be abolished by the enzyme cleaved linker, which weakens its binding affinity to the MHC-I protein.

In some embodiments the β2m polypeptide comprises or consists of the amino acid sequence set forth in SEQ ID NO: 9.

In some embodiments the class I heavy chain polypeptide is comprised or consists of an HLA-A, HLA-B, HLA-C, 1a, 1b, H-2-K, H-2-Dd or H-2-Ld heavy chain.

In some embodiments the MHC-I heavy chain polypeptide comprises or consists of the amino acid sequence set forth in SEQ ID NO: 10, SEQ ID NO: 11 or SEQ ID NO: 12.

In some embodiments the fusion protein further comprises a GS-linker having the amino acid sequence PGS preceding a BirA motif having the amino acid sequence set forth in SEQ ID NO: 13 at the C-terminal end of the MHC-I heavy chain polypeptide, for streptavidin tetramerization and, preferably, also a His6× peptide having the amino acid sequence set forth in SEQ ID NO: 14.

In some embodiments the fusion protein comprises or consists of an amino acid sequence selected from the group comprising or consisting of SEQ ID NO: 15, SEQ ID NO: 16, SEQ ID NO: 17, SEQ ID NO: 18 and SEQ ID NO: 19.

According to a second aspect of the present invention, there is provided a complexed multimer of the single chain fusion protein of any aspect of the invention, wherein the multimer comprises or consists of a plurality of said single chain fusion protein.

In some embodiments the complexed multimer comprises or consists of a dimer, trimer, tetramer or pentamer of said single chain fusion protein.

In some embodiments said single chain fusion proteins are complexed with, for example, streptavidin or other complexing agent. In preferred embodiments the single chain fusion proteins are complexed with streptavidin.

According to a third aspect of the present invention, there is provided an isolated recombinant DNA molecule comprising or consisting of a DNA sequence encoding a single chain fusion protein of any one of claims 1 to 17.

In some embodiments the DNA sequence further encodes a secretion leader polypeptide, preferably a secretion leader polypeptide such as Mellitin comprising or consisting of the amino acid sequence set forth in SEQ ID NO: 1.

It would be understood that due to the redundancy in the genetic code, a nucleic acid sequence may have less than 100% identity and still encode the same amino acid sequence.

In some embodiments the DNA sequence encoding the secretion leader of Mellitin has, due to redundancy in the genetic code, at least 85%, at least 90%, at least 95% or 100% nucleic acid sequence identity to the nucleic acid sequence set forth in SEQ ID NO: 20.

In some embodiments the DNA sequence encodes for a secretion leader polypeptide, a peptide ligand and a first linker polypeptide with a combined length of about 60 amino acids or less, preferably of about 55 amino acids or less, more preferably of about 50 amino acids.

In some embodiments the combined length is 50 amino acids.

In some embodiments the DNA sequence encoding the first linker has, due to redundancy in the genetic code, at least 85%, at least 90%, at least 95% or 100% nucleic acid sequence identity to the nucleic acid sequence set forth in SEQ ID NO: 21.

In some embodiments the DNA sequence encoding the second linker has, due to redundancy in the genetic code, at least 85%, at least 90%, at least 95% or 100% nucleic acid sequence identity to the nucleic acid sequence set forth in SEQ ID NO: 22.

In some embodiments the DNA sequence encoding the peptide ligand has, due to redundancy in the genetic code, at least 85%, at least 90%, at least 95% or 100% nucleic acid sequence identity to the nucleic acid sequence set forth in SEQ ID NO: 23, SEQ ID NO: 24, SEQ ID NO: 25, SEQ ID NO: 26 or SEQ ID NO: 27.

In some embodiments the DNA sequence encoding the β2m polypeptide has, due to redundancy in the genetic code, at least 85%, at least 90%, at least 95% or 100% nucleic acid sequence identity to the nucleic acid sequence set forth in SEQ ID NO: 28.

In some embodiments the DNA sequence encoding the MHC-I heavy chain polypeptide has, due to redundancy in the genetic code, at least 85%, at least 90%, at least 95% or 100% nucleic acid sequence identity to the nucleic acid sequence set forth in SEQ ID NO: 29, SEQ ID NO: 30 or SEQ ID NO: 31.

In some embodiments the DNA sequence encoding the third GS-linker has, due to redundancy in the genetic code, at least 85%, at least 90%, at least 95% or 100% nucleic acid sequence identity to the nucleic acid sequence CCGGGTAGT and/or the DNA sequence encoding the BirA motif has, due to redundancy in the genetic code, at least 85%, at least 90%, at least 95% or 100% nucleic acid sequence identity to the nucleic acid sequence set forth in SEQ ID NO: 32 and/or the DNA sequence encoding the His6× peptide has, due to redundancy in the genetic code, at least 85%, at least 90%, at least 95% or 100% nucleic acid sequence identity to the nucleic acid sequence set forth in SEQ ID NO: 33.

In some embodiments the DNA sequence encoding the fusion protein has, due to redundancy in the genetic code, at least 85%, at least 90%, at least 95% or 100% nucleic acid sequence identity to the nucleic acid sequence set forth in SEQ ID NO: 34, SEQ ID NO: 35, SEQ ID NO: 36, SEQ ID NO: 37 or SEQ ID NO: 38.

According to a fourth aspect of the present invention, there is provided an expression vector comprising the recombinant DNA molecule defined in any aspect of the invention.

According to a fifth aspect of the present invention, there is provided use of an expression vector according to the third aspect for the recombinant production of secreted fusion proteins.

According to a sixth aspect of the present invention, there is provided a method for the production of recombinant secreted fusion proteins, wherein the fusion proteins are defined in the first aspect, comprising the steps:

(i) cultivating a eukaryotic or prokaryotic cell that has been transfected with a recombinant DNA molecule as defined in the second aspect, or an expression vector defined in the third aspect in a cultivation medium and

(ii) recovering the recombinant secreted fusion proteins from the cell or the cultivation medium.

In some embodiments the cell is a eukaryotic cell and has been infected with a recombinant baculovirus expressing said expression vector. In some embodiments the eukaryotic cell is a mammalian cell. In some embodiments the mammalian cell is Chinese hamster ovary cell or human kidney cell.

In some embodiments the eukaryotic cell is an insect cell. In some embodiments the insect cell is preferably a Sf9 or Sf21 cell from Spodoptera frugiperda or Hi5 cell from Trichoplusia ni.

According to a seventh aspect of the present invention, there is provided a method of defining a peptide ligand suitable for successful production of a single chain fusion protein for peptide exchange according to the first aspect, comprising the steps:

i) identify an antigenic or non-antigenic peptide, known to bind to a MHC-I protein of the single chain fusion protein, of from about 4 to 30 amino acid residues, from about 6 to 20 amino acid residues or preferably from about 8 to 15 amino acid residues in length, and its amino acid sequence;

(ii) modify one or more of the amino acids of the peptide that are not interacting with a HLA binding groove of the MHC-1 protein until the peptide sequence, while retaining a HLA binding motif, does not map to any known antigen and/or is not recognized by T cells anymore, wherein preferably the modified amino acids are mutated to or substituted by neutral non-polar residues such as alanine and are exposed out of the HLA binding groove;

(iii) produce monomers of the single chain fusion protein and verify proper binding of peptide ligand to MHC-I based on monomer secretion.

In some embodiments the peptide ligand is a stabilizing ligand.

According to an eighth aspect of the present invention, there is provided a method of peptide exchange comprising the steps:

i) providing a fusion protein of the first aspect or a complexed multimer of the second aspect;

ii) co-incubating, for a period of time, the fusion protein or complexed multimer with a peptide of interest and a cleavage enzyme that will cleave said first linker polypeptide, independent of the order of the components added, wherein said cleavage results in exchange of the peptide ligand with the peptide of interest, thereby generating a distinctively-labeled, soluble MHC-I monomer or complexed multimer loaded with the peptide of interest.

In some embodiments the peptide of interest is a peptide corresponding to viral-derived epitope.

In some embodiments step ii) is performed in an exchange buffer between about pH 5 and about pH 8.0, for a period of about 1 h to about 16 h, at a temperature of between about 15° C. to about 37° C. to release the peptide ligand and allow rescue peptide binding.

According to a ninth aspect of the present invention, there is provided use of a fusion protein of the first aspect or a complexed multimer of the second aspect to detect, isolate or manipulate antigen-specific CD8⁺ T cells.

In some embodiments the complexed multimer is a tetramer.

In some embodiments the antigen-specific CD8⁺ T cells are specific for a particular antigenic peptide derived from viruses, bacteria and self-antigen or tumours.

### BRIEF DESCRIPTION OF THE FIGURES

FIGS. 1A-B show the experimental design and results for the validation of enzyme exchangeable HLA-A*11:01 with exchangeable peptide AVFAAASDAK (SEQ ID NO: 4). FIG. 1A shows a table of the experimental design for the validation of enzyme exchangeable HLA-A*11:01 with exchangeable peptide AVFAAASDAK (SEQ ID NO: 4) for tetramer construction and binding to antigen-specific CD8⁺ T cells. FIG. 1B shows the results. Peptides corresponding to viral-derived epitopes were loaded into the new HLA construct after enzyme-mediated cleavage of the stabilizing peptide; they were also loaded into classical HLA monomer constructs using standard UV exchange protocols. The two types of monomers were then tetramerized and tested on PBMC from healthy donor for detection of peptide-specific CD8⁺ T cells and also level of background staining. The donor PBMC contains peptide 4 and peptide 5-specific CD8⁺ T cells. Data shown above are gated on live CD45⁺CD3⁺CD8⁺ cells. The new HLA-A*11:01 construct with exchangeable peptide AVFAAASDAK (SEQ ID NO: 4) allows an efficient peptide exchange after enzymatic cleavage of the stabilizing peptide, as the level of detection of peptide-specific T cells is comparable to that obtained with the UV-exchanged construct (0.49-0.75% versus 0.5-0.76% of CD8⁺ T cells, respectively).

FIG. 2 shows the experimental design and results for the validation of enzyme exchangeable HLA-A*11:01 with exchangeable peptide AVFAAASDAK (SEQ ID NO: 4) for multiplexed tetramer staining on mass cytometer. (A) UV exchangeable and enzyme exchangeable HLA-A*11:01 were loaded with peptides shown in (A). Triple coded tetramers were formed. Streptavidin codes are as shown in the table (A). Healthy HLA-A*11:01 specific PBMCs were stained with a cocktail of multiplexed-triple coded HLA-A*11:01 tetramer, anti-CD45, anti-CD3, anti-CD8 and live-dead staining followed by acquiring on mass cytometer. (B) Data shown are gated on Live⁺CD45⁺CD3⁺CD8⁺ cells. Dot plot indicates EBV peptide IVTDFSVIK (SEQ ID NO: 39)-specific CD45⁺CD3⁺CD8⁺ cells. (C) Data shown are gated on Live⁺CD45⁺CD3⁺CD8⁺ cells. Dot plot indicates EBV peptide NTLEQTVKK (SEQ ID NO: 40)-specific CD45⁺CD3⁺CD8⁺ cells. (D) Data shown are gated on Live⁺CD45⁺CD3⁺CD8⁺ cells. Dot plot indicates Influenza peptide AVFDRKSDAK (SEQ ID NO: 5) specific CD45⁺CD3⁺CD8⁺ cells. Overall, New HLA-A*11:01 construct with exchangeable peptide AVFAAASDAK (SEQ ID NO: 4) allows an efficient peptide exchange after enzymatic cleavage of the stabilizing peptide, as well as allow triple coded-multiplexed tetramer staining. The level of detection of EBV peptide 1, EBV peptide 2 and Influenza Peptide 1 specific T cells is comparable to that obtained with the UV-exchanged construct. (0.74% versus 0.66% of CD8⁺ T cells for EBV peptide 1, 0.027% versus 0.03% of CD8⁺ T cells for EBV Peptide 2 and 0.68% versus 0.5% of CD8⁺ T cells for Influenza Peptide 1, respectively)

FIGS. 3A-B show the experimental design and results for the validation of enzyme exchangeable HLA-A*02:01 with exchangeable peptide KILGRVFFV (SEQ ID NO: 6) for tetramer construction and binding to antigen-specific CD8⁺ T cells. Peptide 1, 2, 3 and 15 corresponding to viral-derived epitopes were loaded into the new HLA construct after enzyme-mediated cleavage of the stabilizing peptide using 4 different exchange protocols as indicated; Peptides were also loaded into classical HLA monomer constructs using standard UV exchange protocol. The various monomers were then tetramerized and tested on PBMC from healthy donor for detection of peptide-specific CD8⁺ T cells and also level of background staining. The donor PBMC contains peptide 1-specific CD8⁺ T cells. Data shown are gated on live CD45⁺CD3⁺CD8⁺ cells. Overall, the new construct allows an efficient peptide exchange after enzymatic cleavage of the stabilizing peptide; the best protocols for enzymatic exchange are pH 6.2 cacodylate buffer at RT and pH 8 TRIS buffer at RT, as they allow for high resolution of the peptide-specific CD8⁺ T cell population. The level of detection of peptide 1-specific CD8⁺ T cells is comparable to that obtained with the UV-exchanged tetramers (1.14-1.34% versus 0.98-1.21% of CD8⁺ T cells, respectively).

FIGS. 4A-B show the experimental design and results for the validation of enzyme exchangeable HLA-A*02:01 with exchangeable peptide KLAEAIFKL (SEQ ID NO: 7) for tetramer construction and binding to antigen-specific CD8⁺ T cells. Peptide 1, 2, and 11 corresponding to viral-derived epitopes were loaded into the new HLA construct after enzyme-mediated cleavage of the stabilizing peptide using 2 different exchange protocols as indicated; Peptides were also loaded into classical HLA monomer constructs using standard UV exchange protocol. The various monomers were then tetramerized and tested on PBMC from healthy donor for detection of peptide-specific CD8⁺ T cells and also level of background staining. The donor PBMC contains peptide 1-specific CD8⁺ T cells. Data shown are gated on live CD45⁺CD3⁺CD8⁺ cells. Overall, the new HLA-A*02:01 construct with exchangeable peptide KLAEAIFKL (SEQ ID NO: 7) allows efficient peptide exchange after enzymatic cleavage of the stabilizing peptide; the best protocols for enzymatic exchange are pH 6.2 cacodylate buffer at RT and pH 8 TRIS buffer at RT, as they allow for high resolution of the peptide-specific CD8⁺ T cell population. The level of detection of peptide 1-specific CD8⁺ T cells is comparable to that obtained with the UV-exchanged tetramers (0.92-1.38% versus 1.14-1.26% of CD8⁺ T cells, respectively).

FIG. 5 shows the experimental design and results for the validation of enzyme exchangeable HLA-A*02:01 with exchangeable peptide KLAEAIFKL (SEQ ID NO: 7) for multiplexed tetramer staining on mass cytometer. UV exchangeable and enzyme exchangeable HLA-A*02:01 were loaded with different peptides as shown in figure (A). Triple coded tetramers were formed. Streptavidin codes are as shown in the table (A). Healthy HLA-A*02:01 PBMCs were stained with a cocktail of multiplexed-triple coded HLA-A*02:01 tetramers, anti-CD45, anti-CD3, anti-CD8⁺ and live-dead staining followed by acquiring on mass cytometer (B) Data shown are gated on Live⁺CD45⁺CD3⁺CD8⁺ cells. Dot plot indicates CMV peptide NLVPMVATV (SEQ ID NO: 41)-specific CD45⁺CD3⁺CD8⁺ cells. Overall, New HLA-A*02:01 construct with exchangeable peptide KLAEAIFKL (SEQ ID NO: 7) allows an efficient peptide exchange after enzymatic cleavage of the stabilizing peptide, as well as allow triple coded-multiplexed tetramer staining. The level of detection of CMV peptide 1 specific T cells is comparable to that obtained with the UV-exchanged construct (1.28% versus 1.08% for CMV peptide 1).

FIG. 6 shows results for the validation of enzyme exchangeable HLA-A*01:01 with exchangeable peptide YAETAAFAY (SEQ ID NO: 8) for tetramer construction and binding to antigen-specific CD8⁺ T cells. CMV-derived peptide VETEHDTLLY (SEQ ID NO: 42) was loaded into the new HLA construct after enzyme-mediated cleavage of the stabilizing peptide YAETAAFAY (SEQ ID NO: 8); it was also loaded into classical HLA monomer constructs using standard UV exchange protocols. The two types of monomers were then tetramerized with streptavidin and tested on PBMC from healthy donor for detection of CMV peptide VETEHDTLLY (SEQ ID NO: 42)-specific CD8⁺ T cells and also level of background staining. Data shown above are gated on live CD45⁺CD3⁺ cells. The new HLA-A*01:01 construct with exchangeable peptide YAETAAFAY (SEQ ID NO: 8) allows an efficient peptide exchange after enzymatic cleavage of the stabilizing peptide, as the level of detection of peptide-specific T cells is comparable to that obtained with the UV-exchanged construct (1.25% versus 1.5% of CD8⁺ T cells, respectively).

### DEFINITIONS

Certain terms employed in the specification, examples and appended claims are collected here for convenience.

As used in the specification and the appended claims, the singular forms “a,” “an” and “the” include plural referents unless the context clearly dictates otherwise.

As used herein, ranges can be expressed as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that each unit between two particular units are also disclosed. For example, if 10 and 15 are disclosed, then 11, 12, 13, and 14 are also disclosed.

The terms “amino acid” or “amino acid sequence,” as used herein, refer to an oligopeptide, peptide, polypeptide, or protein sequence, or a fragment of any of these, and to naturally occurring or synthetic molecules. Where “amino acid sequence” is recited herein to refer to an amino acid sequence of a naturally occurring protein molecule, “amino acid sequence” and like terms are not meant to limit the amino acid sequence to the complete native amino acid sequence associated with the recited protein molecule.

As used herein, the terms “polypeptide”, “peptide” or “protein” refer to one or more chains of amino acids, wherein each chain comprises amino acids covalently linked by peptide bonds, and wherein said polypeptide or peptide can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. A “polypeptide”, “peptide” or “protein” can comprise one (termed “a monomer”) or a plurality (termed “a multimer”) of amino acid chains.

As used herein, the term “stabilizing polypeptide” refers to an inert polypeptide which binds and/or associates with the antigen binding pocket of a MHC-I protein, thereby ensuring the conformation of the MHC-I protein until the stabilizing peptide can be cleaved off and replaced by a peptide of interest. It is understood that the stabilizing peptide may be antigenic or non-antigenic and any antigenicity of the peptide ligand may be abolished by the enzyme cleaved linker, which weakens its binding affinity to the MHC-I protein. In one example, the stabiliser protein may be cleaved off and replaced or exchanged by a peptide corresponding to viral-derived epitope.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

### DETAILED DESCRIPTION OF THE INVENTION

Bibliographic references mentioned in the present specification are for convenience listed at the end of the examples. The whole content of such bibliographic references is herein incorporated by reference.

### EXAMPLES

A person skilled in the art will appreciate that the present invention may be practiced without undue experimentation according to the methods given herein. The methods, techniques and chemicals are as described in the references given or from protocols in standard biotechnology and molecular biology text books. Standard molecular biology techniques known in the art and not specifically described were generally followed as described in Sambrook and Russel, Molecular Cloning: A Laboratory Manual, Cold Springs Harbor Laboratory, New York (2001). Briefly, a melittin-leader bearing baculovirus expression vector is used to make the pMHC-I single chain trimer protein. In the single chain trimer design, the single chain module is separated by the first and second linkers, where the N-terminus stabilizing peptide bears an additional cleavable DDDD|K peptide sequence (SEQ ID NO: 43) to aid peptide dissociation and exchange. Following standard bacmid preparation and infection of insect cells, different single chain pMHC-I proteins are overexpressed as secreted proteins in Sf9 (Spodoptera frugiperda) or High Five (Trichoplusia ni.) cells. The secreted single chain pMHC-I proteins are purified, biotinylated and validated for tetramer staining of CD8⁺ T cells using flow and mass cytometry.

### Materials and Methods

### Engineering, Production and Purification of MHC-1 Single Chain Protein

A melittin-leader bearing baculovirus expression vector such as the PFASTBAC™1 vector (ThermoFisher) is used to make the secreted peptide-MHC (pMHC)-I single chain trimer protein. Following standard molecular cloning techniques, the gene encoding the single chain trimer is subcloned into the baculovirus expression vector. In the single chain trimer design in the order of peptide, β2m and MHC-I heavy chain polypeptide, the single chain module is separated by the first and second linkers, where the N-terminus stabilizing peptide bears an additional cleavable DDDD|K peptide sequence (SEQ ID NO: 43) to aid peptide dissociation and exchange upon enterokinase addition. Following standard bacmid preparation and infection of insect cells, different single chain pMHC-I proteins are overexpressed as secreted proteins in Sf9 (Spodoptera frugiperda) or HIGH FIVE™ (Trichoplusia ni; BTI-Tn-5B1-4) cells using the BAC-TO-BAC™ Baculovirus Expression System (ThermoFisher). The secreted single chain pMHC-I proteins are purified from the culture media using the standard Ni-NTA resin purification in 20 mM Tris pH 8.0 and 150 mM NaCl buffer, eluted with imidazole and biotinylated with BirA enzyme as previously described (Fairhead M. and Howarth M. Methods Mol Biol. 1266: 171-84 (2015); incorporated herein by reference). The biotinylated protein is further purified in size exclusion chromatography using the SUPERDEX™ S200 gel filtration column in 20 mM Tris pH 8.0 and 150 mM NaCl buffer.

### Peptide Exchange

UV-cleavable pMHC molecules are produced in house as previously described (Newell E W, et al., Nat Biotechnol. (7): 623-9 (2013); Leong M L, and Newell E W. Methods Mol Biol. 1346: 115-31 (2015); incorporated herein by reference). For UV-mediated peptide exchange, UV cleavable pMHC-I are exposed to 365 nm UV irradiation for 15-min in the presence of a single peptide of interest. Peptide exchange reactions are set up in 96 well plates at least 12 h before tetramerization. For enzyme mediated peptide exchange, 0.17 μl of enterokinase (16,000 U/μl) (NEB cat no. P8070S) is added to 10 μl of 0.1 mg/ml enzyme-cleavable MHC-I single chain protein. Enzyme reaction is set at either pH 6 or pH 8 for at least 16 h at 37° C. or at room temperature (RT). To quench the enzyme reaction, a triple volume of 1×PBS is added followed by 1 h incubation in ice.

### Tetramerization

APC, PE and APC-Cy7-labelled streptavidins for flow cytometry staining are purchased from BioLegend (San Diego, Calif., USA). For mass cytometry, streptavidin is produced in-house as described in (Newell E W, et al., Nat Biotechnol. (7): 623-9 (2013); incorporated herein by reference) and conjugated with heavy metals using DN3 polymer linker as reported previously (Leong M L, and Newell E W. Methods Mol Biol. 1346: 115-31 (2015); incorporated herein by reference). To produce pMHC tetramers, fluorescently or heavy metal labelled streptavidin or streptavidin mixtures are added to the respective pMHC complexes in three repeated steps (10 min incubation at RT per addition) so to achieve a total final molar ratio of 1 (streptavidin): 4 (pMHC). 10 μM free biotin (Sigma) is added to each reaction to quench free streptavidin molecules. In case of multiplexing experiments, tetramerized pMHC complexes are pulled together and concentrated by using a 10 kDa cut-off filter (Merck Millipore), buffer is exchanged with cytometry buffer (PBS, 2% v/v FCS, 2 mM EDTA, 0.05% v/v sodium azide), filtered through a 1 μm centrifugal filter (Merck Millipore) and used to stain the cells.

### Cell Staining

For pMHC tetramer staining experiments, PBMC samples from HLA-A*02:01, HLA-A*11:01 and HLA-A*01:01 positive healthy donors are used. For mass cytometry and flow cytometry, cells are stained with tetramers and surface antibodies as described earlier (Fehlings M, et al., J Immunol Methods. 453: 30-36 (2018); Simoni Y, et al., Methods Mol Biol. 1989: 147-158 (2019), incorporated herein by reference). Briefly, for mass cytometry, purified antibodies are tagged with heavy metal loaded maleimide conjugated DN3 MAXPAR® chelating polymers (Fluidigm) according to manufacturer's recommendations. Three to 5 million cells are transferred to 96 well plates and stained with 200 μM cisplatin (Sigma) for live/dead discrimination. 100 μl of tetramer cocktail is added to cisplatin-stained cells and incubated for 1 hour at RT followed by staining with 50 μl of heavy metal-labeled antibody cocktail for 30 min on ice. Stained cells are fixed overnight in 2% v/v paraformaldehyde (Electron Microscopy Sciences) at 4° C. followed by labelling with 250 nM (1:2000) iridium DNA nucleic acid intercalator (Fluidigm) in 2% v/v PFA in 1×PBS at room temperature for 20 min. Finally, cells are washed and resuspended in distilled water at 0.5 million cells/ml cell concentration. For flow cytometry, 0.5 to 1 million cells are transferred to 96 well plates and stained with 100 μl of tetramer cocktail for 1 hour at RT, followed by staining with a cocktail of BV421 labelled anti-human CD45 (clone HI30), Alexa Fluor 700 labelled anti-human CD3 (clone OKT3), BV605 or FITC labelled anti-human CD8 (clone SK1) and LIVE/DEAD® fixable Aqua Dead Cell Stain (ThermoFisher) for the live/dead discrimination for 30 min on ice. Stained cells are resuspended in 1×PBS.

### Data Analysis

Fluorescently labelled samples are acquired by BD LSRFORTESSA™ and heavy metal labelled samples are acquired an a Fluidigm HELIOS™ mass cytometer. Mass cytometry data is converted to .fcs format by Fluidigm acquisition software. The signal of each parameter is normalized based on the EQ beads (Fluidigm) as described previously (Finck R, et al., Cytometry A. 83(5): 483-94 (2013), incorporated herein by reference). FLOWJO™ software is used for flow as well as mass cytometry data analysis. For Flow data, CD8⁺ T cells are selected by manually gating on Live⁺CD45⁺CD3⁺CD8⁺ cells. For mass cytometry data, CD8⁺ T cells are selected by manually gating on cisplatin CD45⁺CD3⁺CD8⁺. Tetramer positive cell populations are then identified based on the fluorescently- or heavy metal-labelled streptavidin assigned to the pMHC tetramer.

### Example 1

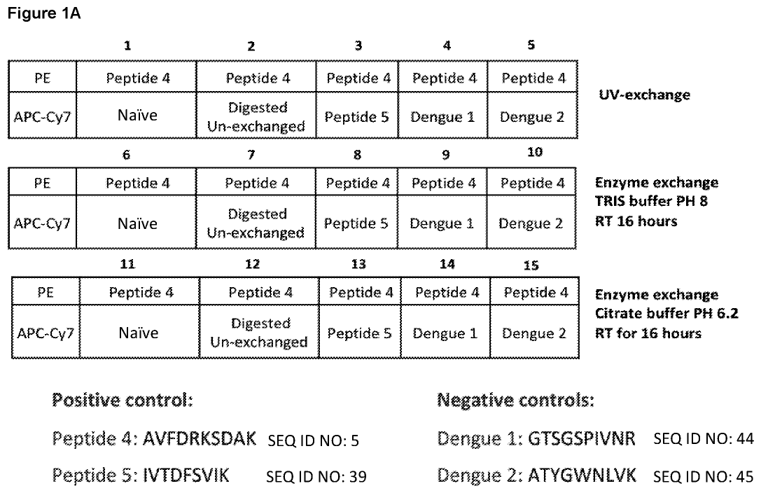

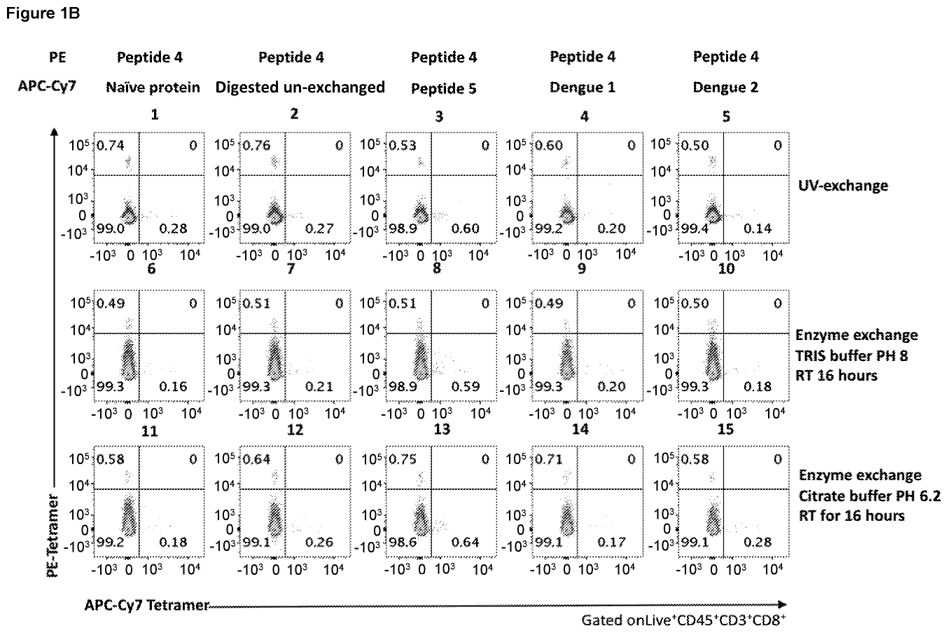

Peptides corresponding to viral-derived epitopes were loaded into the new HLA construct after enzyme-mediated cleavage of the stabilizing peptide; they were also loaded into classical HLA monomer constructs using standard UV exchange protocols described above. The two types of monomers were then tetramerized and tested on PBMC from healthy donor for detection of peptide-specific CD8⁺ T cells and also level of background staining. The donor PBMC contains peptide 4 and peptide 5-specific CD8⁺ T cells. FIGS. 1A and 1B show the experimental design and results for the validation of enzyme exchangeable HLA-A*11:01 with exchangeable peptide AVFAAASDAK (SEQ ID NO: 4). FIG. 1A shows a table of the experimental design for the validation of enzyme exchangeable HLA-A*11:01 with exchangeable peptide AVFAAASDAK (SEQ ID NO: 4) for tetramer construction and binding to antigen-specific CD8⁺ T cells. FIG. 1B shows the results. Data shown above are gated on live CD45⁺CD3⁺CD8⁺ cells. The new HLA-A*11:01 construct with exchangeable peptide AVFAAASDAK (SEQ ID NO: 4) allows an efficient peptide exchange after enzymatic cleavage of the stabilizing peptide, as the level of detection of peptide-specific T cells is comparable to that obtained with the UV-exchanged construct (0.49-0.75% versus 0.5-0.76% of CD8⁺ T cells, respectively).

### Example 2

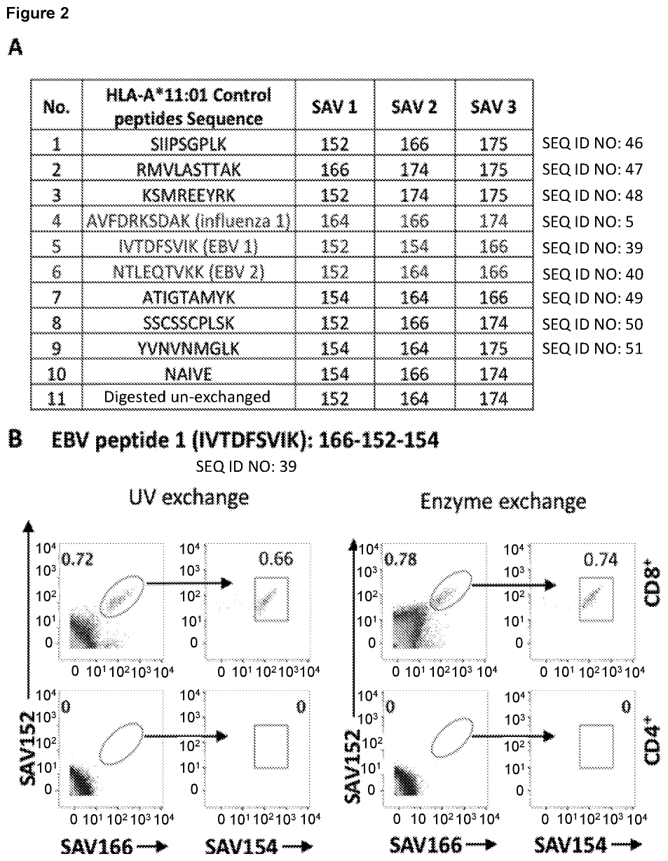

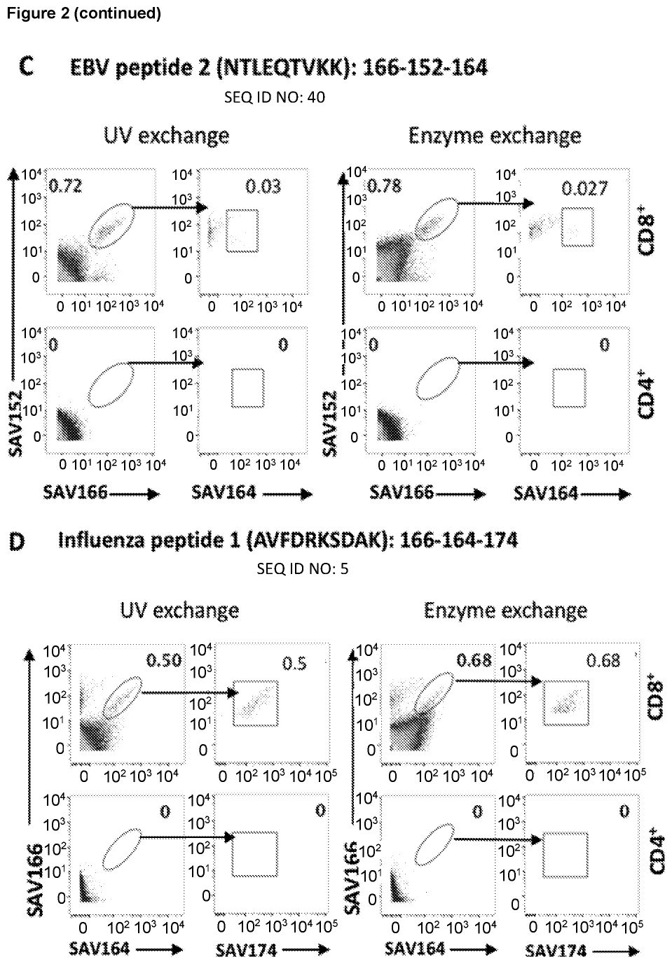

The experimental design and results for validation of enzyme exchangeable HLA-A*11:01 with exchangeable peptide AVFAAASDAK (SEQ ID NO: 4) for multiplexed tetramer staining on mass cytometer are shown in FIG. 2. Methods for exchange are described above. UV exchangeable and enzyme exchangeable HLA-A*11:01 were loaded with peptides shown in (A). Triple coded tetramers were formed. Streptavidin codes are as shown in the table. Healthy HLA-A*11:01 specific PBMCs were stained with a cocktail of multiplexed-triple coded HLA-A*11:01 tetramer, anti-CD45, anti-CD3, anti-CD8 and live-dead staining followed by acquiring on mass cytometer using methods described above. The FIG. 2B data shown are gated on Live⁺CD45⁺CD3⁺CD8⁺ cells, and the Dot plot indicates EBV peptide IVTDFSVIK (SEQ ID NO: 39)-specific CD45⁺CD3⁺CD8⁺ cells. The FIG. 2C data shown are gated on Live⁺CD45⁺CD3⁺CD8⁺ cells, and the Dot plot indicates EBV peptide NTLEQTVKK (SEQ ID NO: 40)-specific CD45⁺CD3⁺CD8⁺ cells. The FIG. 2B data shown are gated on Live⁺CD45⁺CD3⁺CD8⁺ cells, and the Dot plot indicates Influenza peptide AVFDRKSDAK (SEQ ID NO: 5) specific CD45⁺CD3⁺CD8⁺ cells. Overall, the new HLA-A*11:01 construct with exchangeable peptide AVFAAASDAK (SEQ ID NO: 4) allows an efficient peptide exchange after enzymatic cleavage of the stabilizing peptide, as well as allows triple coded-multiplexed tetramer staining. The level of detection of EBV peptide 1, EBV peptide 2 and Influenza Peptide 1 specific T cells is comparable to that obtained with the UV-exchanged construct. (0.74% versus 0.66% of CD8⁺ T cells for EBV peptide 1, 0.027% versus 0.03% of CD8⁺ T cells for EBV Peptide 2 and 0.68% versus 0.5% of CD8⁺ T cells for Influenza Peptide 1, respectively)

### Example 3

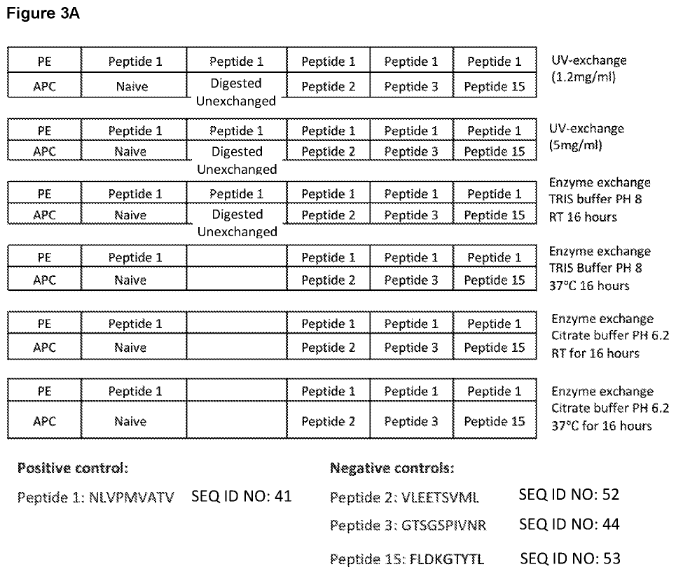

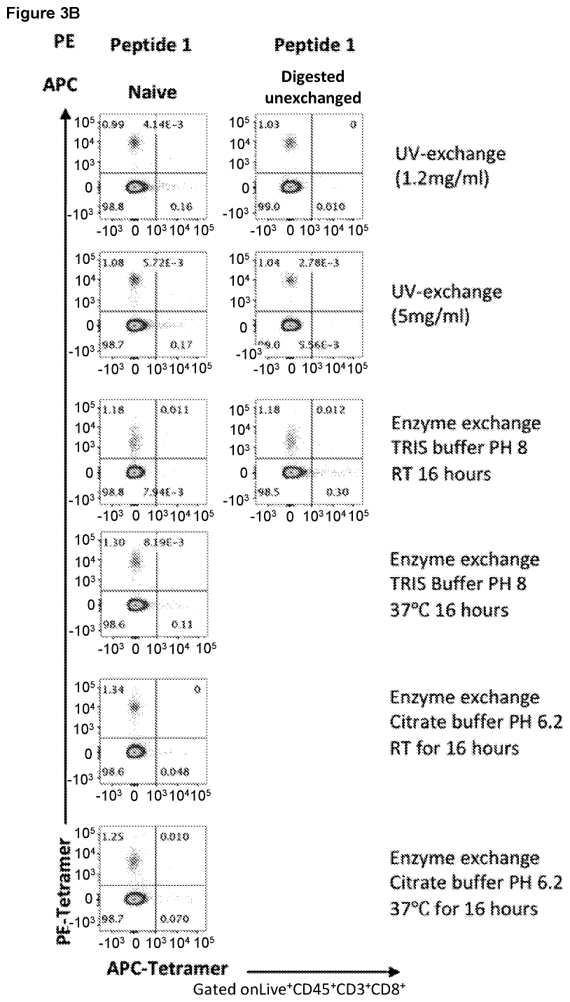

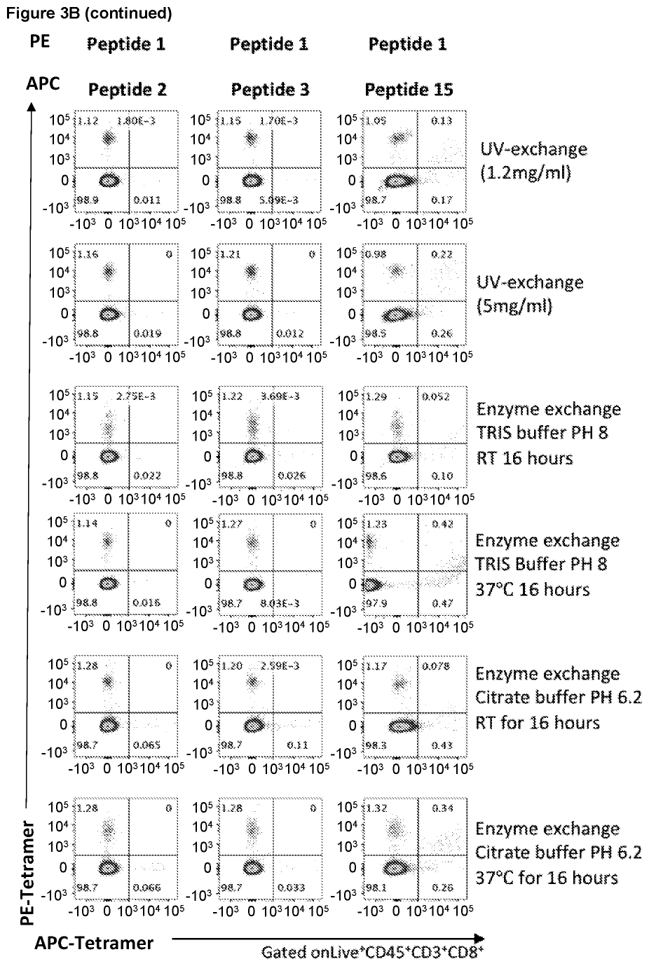

The experimental design and results for validation of enzyme exchangeable HLA-A*02:01 with exchangeable peptide KILGRVFFV (SEQ ID NO: 6) for tetramer construction and binding to antigen-specific CD8⁺ T cells are shown in FIG. 3. Peptides 1, 2, 3 and 15, corresponding to viral-derived epitopes, were loaded into the new HLA construct after enzyme-mediated cleavage of the stabilizing peptide using 4 different exchange protocols as indicated; Peptides were also loaded into classical HLA monomer constructs using standard UV exchange protocol described above. The various monomers were then tetramerized as described above and tested on PBMC from healthy donor for detection of peptide-specific CD8⁺ T cells and also level of background staining. The donor PBMC contains peptide 1-specific CD8⁺ T cells. The data shown in FIG. 3B are gated on live CD45⁺CD3⁺CD8⁺ cells. Overall, the new construct allows an efficient peptide exchange after enzymatic cleavage of the stabilizing peptide; the best protocols for enzymatic exchange are pH 6.2 cacodylate buffer at RT and pH 8 TRIS buffer at RT, as they allow for high resolution of the peptide-specific CD8⁺ T cell population. The level of detection of peptide 1-specific CD8⁺ T cells is comparable to that obtained with the UV-exchanged tetramers (1.14-1.34% versus 0.98-1.21% of CD8⁺ T cells, respectively).

### Example 4

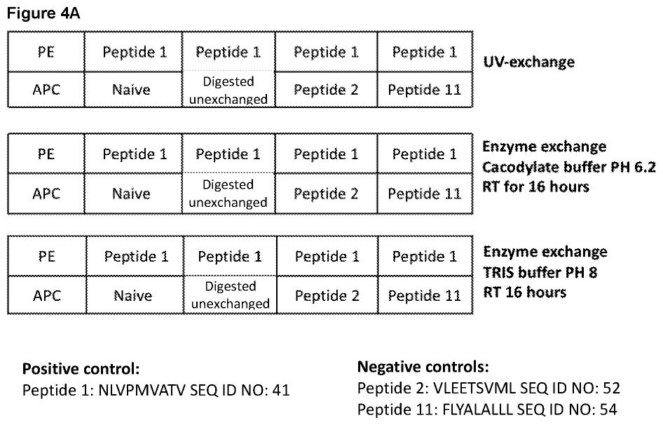

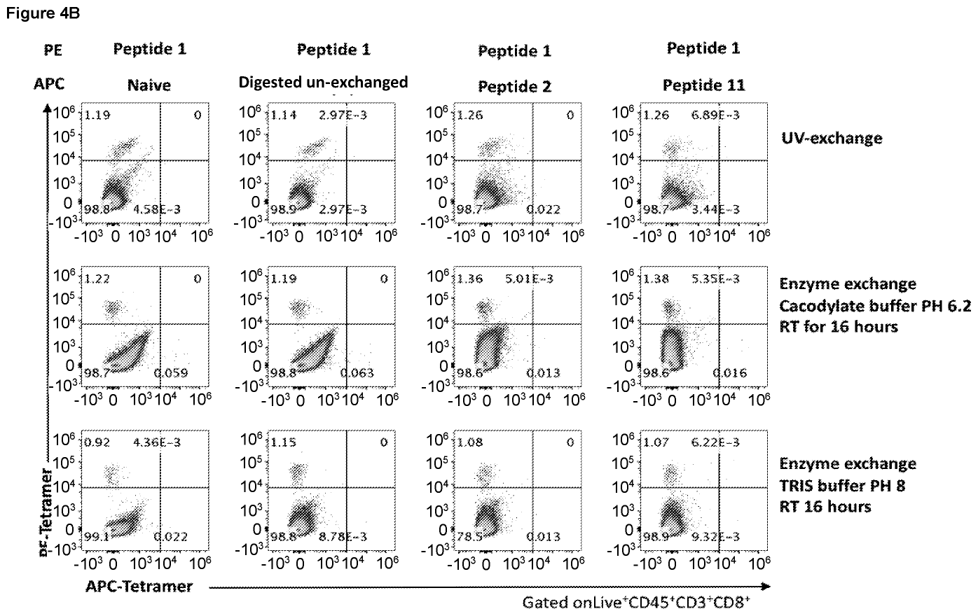

The experimental design and results for validation of enzyme exchangeable HLA-A*02:01 with exchangeable peptide KLAEAIFKL (SEQ ID NO: 7) for tetramer construction and binding to antigen-specific CD8⁺ T cells are shown in FIG. 4. Peptides 1, 2, and 11 corresponding to viral-derived epitopes were loaded into the new HLA construct after enzyme-mediated cleavage of the stabilizing peptide using 2 different exchange protocols as indicated;

Peptides were also loaded into classical HLA monomer constructs using standard UV exchange protocol described above. The various monomers were then tetramerized as described above and tested on PBMC from healthy donor for detection of peptide-specific CD8⁺ T cells and also level of background staining. The donor PBMC contains peptide 1-specific CD8⁺ T cells. The data shown in FIG. 4B are gated on live CD45⁺CD3⁺CD8⁺ cells. Overall, the new HLA-A*02:01 construct with exchangeable peptide KLAEAIFKL (SEQ ID NO: 7) allows efficient peptide exchange after enzymatic cleavage of the stabilizing peptide; the best protocols for enzymatic exchange are pH 6.2 cacodylate buffer at RT and pH 8 TRIS buffer at RT, as they allow for high resolution of the peptide-specific CD8⁺ T cell population. The level of detection of peptide 1-specific CD8⁺ T cells is comparable to that obtained with the UV-exchanged tetramers (0.92-1.38% versus 1.14-1.26% of CD8⁺ T cells, respectively).

### Example 5

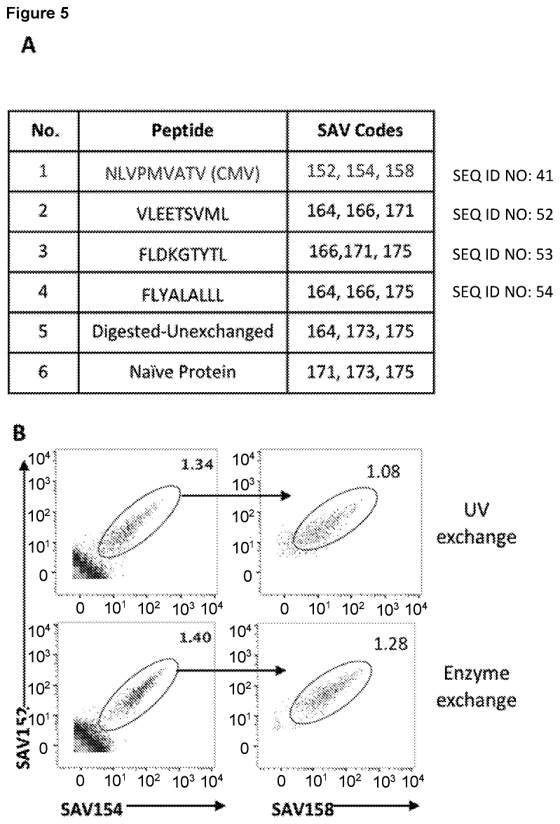

The experimental design and results for the validation of enzyme exchangeable HLA-A*02:01 with exchangeable peptide KLAEAIFKL (SEQ ID NO: 7) for multiplexed tetramer staining on mass cytometer are shown in FIG. 5. UV exchangeable and enzyme exchangeable HLA-A*02:01 were loaded with different peptides as per the method described above, and as shown in FIG. 5A. Triple coded tetramers were formed. Streptavidin codes are as shown in the table. Healthy HLA-A*02:01 PBMCs were stained with a cocktail of multiplexed-triple coded HLA-A*02:01 tetramers, anti-CD45, anti-CD3, anti-CD8⁺ and live-dead staining followed by acquiring on mass cytometer, as described above. Data shown are gated on Live⁺CD45⁺CD3⁺CD8⁺ cells. Dot plot indicates CMV peptide NLVPMVATV (SEQ ID NO: 41)-specific CD45⁺CD3⁺CD8⁺ cells (FIG. 5B). Overall, New HLA-A*02:01 construct with exchangeable peptide KLAEAIFKL (SEQ ID NO: 7) allows an efficient peptide exchange after enzymatic cleavage of the stabilizing peptide, as well as allow triple coded-multiplexed tetramer staining. The level of detection of CMV peptide 1 specific T cells is comparable to that obtained with the UV-exchanged construct (1.28% versus 1.08% for CMV peptide 1).

### Example 6

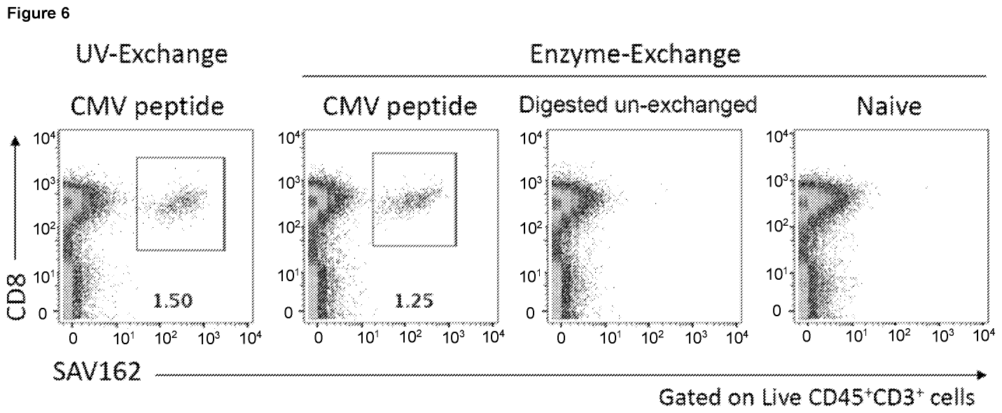

Validation of enzyme exchangeable HLA-A*01:01 with exchangeable peptide YAETAAFAY (SEQ ID NO: 8) for tetramer construction and binding to antigen-specific CD8⁺ T cells is shown in FIG. 6. CMV-derived peptide VETEHDTLLY (SEQ ID NO: 42) was loaded into the new HLA construct after enzyme-mediated cleavage of the stabilizing peptide YAETAAFAY (SEQ ID NO: 8); it was also loaded into classical HLA monomer constructs using standard UV exchange protocols described above. The two types of monomers were then tetramerized with streptavidin as previously described, and tested on PBMC from healthy donor for detection of CMV peptide VETEHDTLLY (SEQ ID NO: 42)-specific CD8⁺ T cells and also level of background staining. Data shown are gated on live CD45⁺CD3⁺ cells. The new HLA-A*01:01 construct with exchangeable peptide YAETAAFAY (SEQ ID NO: 8) allows an efficient peptide exchange after enzymatic cleavage of the stabilizing peptide, as the level of detection of peptide-specific T cells is comparable to that obtained with the UV-exchanged construct (1.25% versus 1.5% of CD8⁺ T cells, respectively).

### REFERENCES

Any listing or discussion of an apparently prior-published document in this specification should not necessarily be taken as an acknowledgement that such document is part of the state of the art or is common general knowledge.
Bakker A H, Hoppes R, Linnemann C, Toebes M, Rodenko B, Berkers C R, Hadrup S R, van Esch W J, Heemskerk M H, Ovaa H, Schumacher T N, Conditional MHC class I ligands and peptide exchange technology for the human MHC gene products HLA-A1, -A3, Proc Natl Acad Sci USA, 105(10): 3825-30 (2008).
Fairhead M. and Howarth M. Site-specific biotinylation of purified proteins using BirA. Methods Mol Biol. 1266:171-84 (2015).
Fehlings M, Chakarov S, Simoni Y, Sivasankar B, Ginhoux F, Newell E W. Multiplex peptide-MHC tetramer staining using mass cytometry for deep analysis of the influenza-specific T-cell response in mice. J Immunol Methods. 453: 30-36 (2018).
Finck R, Simonds E F, Jager A, Krishnaswamy S, Sachs K, Fantl W, Pe'er D, Nolan G P, Bendall S C. Normalization of mass cytometry data with bead standards. Cytometry A. 83(5): 483-94 (2013).
Garboczi D N, Hung D T, Wiley D C. HLA-A2-peptide complexes: refolding and crystallization of molecules expressed in Escherichia coli and complexed with single antigenic peptides. Proc. Natl Acad. Sci. USA 89(8): 3429-3433 (1992).
Leong M L, and Newell E W. Multiplexed Peptide-MHC Tetramer Staining with Mass Cytometry. Methods Mol Biol. 1346: 115-31 (2015).
Newell E W, Sigal N, Nair N, Kidd B A, Greenberg H B, Davis M M. Combinatorial tetramer staining and mass cytometry analysis facilitate T-cell epitope mapping and characterization. Nat Biotechnol. (7): 623-9 (2013).
Simoni Y, Fehlings M, Newell E W. Multiplex MHC Class I Tetramer Combined with Intranuclear Staining by Mass Cytometry. Methods Mol Biol. 1989: 147-158 (2019).

1. A single chain fusion protein comprising, in order from N-terminus to C-terminus of the fusion protein;
(i) a peptide ligand;
(ii) a first linker polypeptide comprising an enzyme-cleavable portion;
(iii) a β-2 microglobulin (β2m) polypeptide;
(iv) a second linker polypeptide; and
(v) a mature Major Histocompatibility Complex Class I (MHC-I) heavy chain polypeptide.

2. The fusion protein of claim 1, wherein the peptide ligand is a stabilizing peptide.

3. The fusion protein of claim 1, wherein the first linker polypeptide comprises preferably about 15, 16, 17, 18, 19, 20 or 21 or more amino acid residues, more preferably about 21 amino acid residues and/or the second linker polypeptide comprises preferably about 15, 16, 17, 18, 19 or 20 or more amino acid residues, more preferably about 20 amino acid residues.

4. The fusion protein of claim 1, wherein the first and second linker polypeptides comprise at least about 80 percent glycine, alanine and/or serine residues.

5. The fusion protein of claim 1, wherein the first linker polypeptide comprises the amino acid sequence set forth in SEQ ID NO: 2 and/or the second linker polypeptide comprises the amino acid sequence set forth in SEQ ID NO: 3.

6. The fusion protein of claim 1, wherein the peptide ligand comprises:
i) from about 4 to 30 amino acid residues; or
ii) from about 6 to 20 amino acid residues; or
iii) from about 8 to 15 amino acid residues; and/or
wherein the peptide ligand is selected from the group comprising the amino acid sequence set forth in SEQ ID NO: 4 (AVFAAASDAK), SEQ ID NO: 5 (AVFDRKSDAK), SEQ ID NO: 6 (KILGRVFFV), SEQ ID NO: 7 (KLAEAIFKL) and SEQ ID NO: 8 (YAETAAFAY).

7. (canceled)

8. The fusion protein of claim 1, wherein the β2m polypeptide comprises the amino acid sequence set forth in SEQ ID NO: 9.

9. The fusion protein of claim 1, wherein the class I heavy chain polypeptide is comprised of an HLA-A, HLA-B, HLA-C, 1a, 1b, H-2-K, H-2-Dd or H-2-Ld heavy chain; and/or wherein the MHC-I heavy chain polypeptide comprises the amino acid sequence set forth in SEQ ID NO: 10, SEQ ID NO: 11 or SEQ ID NO: 12.

10. (canceled)

11. The fusion protein of claim 1, further comprising a GS-linker polypeptide having the amino acid sequence PGS preceding a BirA motif having the amino acid sequence set forth in SEQ ID NO: 13 at the C-terminal end of the MHC-I heavy chain polypeptide, for streptavidin tetramerization and, preferably, also a His6× peptide having the amino acid sequence set forth in SEQ ID NO: 14.

12. The fusion protein of claim 1, wherein the fusion protein comprises the amino acid sequence set forth in SEQ ID NO: 15, SEQ ID NO: 16, SEQ ID NO: 17, SEQ ID NO: 18 or SEQ ID NO: 19.

13. A complexed multimer of the single chain fusion protein of claim 1, wherein the multimer comprises a plurality of said single chain fusion protein.

14. The complexed multimer of claim 13, comprising a dimer, trimer, tetramer or pentamer of said single chain fusion protein.

15. The complexed multimer of claim 13, wherein said single chain fusion proteins are complexed with, for example, streptavidin or other complexing agent.

16. An isolated recombinant DNA molecule comprising a DNA sequence encoding a single chain fusion protein of claim 1.

17. The recombinant DNA molecule of claim 16, wherein the DNA sequence further encodes a secretion leader polypeptide, preferably a secretion leader polypeptide such as Mellitin comprising the amino acid sequence set forth in SEQ ID NO: 1, preferably wherein the DNA sequence encoding the secretion leader of Mellitin has, due to redundancy in the genetic code, at least 85%, at least 90%, at least 95% or 100% nucleic acid sequence identity to the nucleic acid sequence set forth in SEQ ID NO: 20.

18. (canceled)

19. The recombinant DNA molecule of claim 17, wherein the DNA sequence encodes for a secretion leader polypeptide, a peptide ligand and a first linker polypeptide with a combined length of about 60 amino acids or less, preferably of about 55 amino acids or less, more preferably of about 50 amino acids.

20. The recombinant DNA molecule of claim 16, wherein:
the DNA sequence encoding the first linker has, due to redundancy in the genetic code, at least 85%, at least 90%, at least 95% or 100% nucleic acid sequence identity to the nucleic acid sequence set forth in SEQ ID NO: 21; and/or
the DNA sequence encoding the second linker has, due to redundancy in the genetic code, at least 85%, at least 90%, at least 95% or 100% nucleic acid sequence identity to the nucleic acid sequence set forth in SEQ ID NO: 22; and/or
the DNA sequence encoding the peptide ligand has, due to redundancy in the genetic code, at least 85%, at least 90%, at least 95% or 100% nucleic acid sequence identity to the nucleic acid sequence set forth in SEQ ID NO: 23, SEQ ID NO: 24, SEQ ID NO: 25, SEQ ID NO: 26 or SEQ ID NO: 27; and/or
the DNA sequence encoding the β2m polypeptide has, due to redundancy in the genetic code, at least 85%, at least 90%, at least 95% or 100% nucleic acid sequence identity to the nucleic acid sequence set forth in SEQ ID NO: 28; and/or
the DNA sequence encoding the MHC-I heavy chain polypeptide has, due to redundancy in the genetic code, at least 85%, at least 90%, at least 95% or 100% nucleic acid sequence identity to the nucleic acid sequence set forth in SEQ ID NO: 29, SEQ ID NO: 30 or SEQ ID NO: 31.

21. The recombinant DNA molecule of claim 16, wherein the DNA sequence encoding the GS-linker has, due to redundancy in the genetic code, at least 85%, at least 90%, at least 95% or 100% nucleic acid sequence identity to CCGGGTAGT and/or the DNA sequence encoding the BirA motif has, due to redundancy in the genetic code, at least 85%, at least 90%, at least 95% or 100% nucleic acid sequence identity to the nucleic acid sequence set forth in SEQ ID NO: 32 and/or the DNA sequence encoding the His6× peptide has, due to redundancy in the genetic code, at least 85%, at least 90%, at least 95% or 100% nucleic acid sequence identity to the nucleic acid sequence set forth in SEQ ID NO: 33.

22. The recombinant DNA molecule of claim 16, wherein the DNA sequence encoding the fusion protein has, due to redundancy in the genetic code, at least 85%, at least 90%, at least 95% or 100% nucleic acid sequence identity to the nucleic acid sequence set forth in SEQ ID NO: 34, SEQ ID NO: 35, SEQ ID NO: 36, SEQ ID NO: 37 or SEQ ID NO: 38.

23. An expression vector comprising the recombinant DNA molecule defined in claim 16.

24. (canceled)

25. A method for the production of recombinant secreted fusion proteins defined in claim 1 comprising the steps:
i) cultivating a eukaryotic or prokaryotic cell that has been transfected with a recombinant DNA molecule as defined in claim 16, or an expression vector of claim 23 in a cultivation medium and ii) recovering the recombinant secreted fusion proteins from the cell or the cultivation medium.

26. The method according to of claim 25, wherein the cell is a eukaryotic cell and has been infected with a recombinant baculovirus expressing said expression vector, preferably, wherein the eukaryotic cell is a mammalian cell, preferably a Chinese hamster ovary cell or human kidney cell, or the eukaryotic cell is an insect cell, preferably a Sf9 or Sf21 cell from Spodoptera frugiperda or Hi5 cell from Trichoplusia ni.

27. (canceled)

28. A method of defining a peptide ligand suitable for successful production of a single chain fusion protein for peptide exchange comprising the steps:
i) identify an antigenic or non-antigenic peptide, known to bind to a MHC-I protein of the single chain fusion protein, of claim 6, and its amino acid sequence;
ii) modify one or more of the amino acids of the peptide that are not interacting with a HLA binding groove of the MHC-I protein until the peptide sequence, while retaining a HLA binding motif, does not map to any known antigen and/or is not recognized by T cells anymore, wherein preferably the one or more modified amino acids are mutated to or substituted by neutral non-polar residues such as alanine and are exposed out of the HLA binding groove;
iii) produce monomers of the single chain fusion protein and verify proper binding of said peptide ligand to MHC-I based on monomer secretion.

29. A method of peptide exchange comprising the steps:
i) providing a fusion protein of claim 1 or a complexed multimer of claim 13;
ii) co-incubating, for a period of time, the fusion protein or complexed multimer with a peptide of interest and a cleavage enzyme that will cleave said first linker polypeptide, independent of the order of the components added,
wherein said cleavage results in exchange of the peptide ligand with the peptide of interest, thereby generating a distinctively-labeled, soluble MHC monomer or complexed multimer loaded with the peptide of interest.

30. The method of claim 29, wherein step ii) is performed in an exchange buffer between about pH 5 and about pH 8.0, for a period of about 1 h to about 16 h, at a temperature of between about 15° C. to about 37° C. to release the peptide ligand and allow rescue peptide binding.

31. (canceled)


SEQUENCE LISTING (54 sequences)

SEQ ID NO: 1 - protein, 21 aa, Artificial Sequence
  Mellitin secretion leader
MKFLVNVALVFMVVYISYIYA

SEQ ID NO: 2 - protein, 21 aa, Artificial Sequence
  Enterokinase cleavable linker
GGGGSGGGGSGGGGSDDDDKG

SEQ ID NO: 3 - protein, 20 aa, Artificial Sequence
  GS-Linker
GGGGSGGGGSGGGGSGGGGS

SEQ ID NO: 4 - protein, 10 aa, Artificial Sequence
  Peptide ligand 1
AVFAAASDAK

SEQ ID NO: 5 - protein, 10 aa, Artificial Sequence
  Peptide ligand 2
AVFDRKSDAK

SEQ ID NO: 6 - protein, 9 aa, Artificial Sequence
  Peptide ligand 3
KILGRVFFV

SEQ ID NO: 7 - protein, 9 aa, Artificial Sequence
  Peptide ligand 4
KLAEAIFKL

SEQ ID NO: 8 - protein, 9 aa, Artificial Sequence
  Peptide ligand 5
YAETAAFAY

SEQ ID NO: 9 - protein, 99 aa, Artificial Sequence [sequence omitted]

SEQ ID NO: 10 - protein, 273 aa, Artificial Sequence
  A*1101 Heavy chain
HSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 11 - protein, 273 aa, Artificial Sequence
  A*0201 Heavy chain
HSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 12 - protein, 274 aa, Artificial Sequence
  MHC-I heavy chain polypeptide
HSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEL

SEQ ID NO: 13 - protein, 15 aa, Artificial Sequence
  BirA recognition site
LHHILDAQKMVWNHR

SEQ ID NO: 14 - protein, 6 aa, Artificial Sequence
  His6x
HHHHHH

SEQ ID NO: 15 - protein, 468 aa, Artificial Sequence
  A*1101-AVFAAASDAK
MKFLVNVALVFMVVYISYIYAAVFAAASDAKGGGGSGGGGSGGGGSDDDDKGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPGSLHHILDAQKMVWNHRHHHHHH

SEQ ID NO: 16 - protein, 468 aa, Artificial Sequence
  A*1101-AVFDRKSDAK
MKFLVNVALVFMVVYISYIYAAVFDRKSDAKGGGGSGGGGSGGGGSDDDDKGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPGSLHHILDAQKMVWNHRHHHHHH

SEQ ID NO: 17 - protein, 466 aa, Artificial Sequence
  A*0201-KILGRVFFV
MKFLVNVALVFMVVYISYIYAKILGRVFFVGGGGSGGGGSGGGGSDDDDKGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPGSLHHILDAQKMVWNHRHHHHH

SEQ ID NO: 18 - protein, 467 aa, Artificial Sequence
  A*0201-KLAEAIFKL
MKFLVNVALVFMVVYISYIYAKLAEAIFKLGGGGSGGGGSGGGGSDDDDKGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPGSLHHILDAQKMVWNHRHHHHHH

SEQ ID NO: 19 - protein, 467 aa, Artificial Sequence
  A*0101-YAETAAFAY
MKFLVNVALVFMVVYISYIYAYAETAAFAYGGGGSGGGGSGGGGSDDDDKGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPGSLHHILDAQKMVWNHRHHHHHH

SEQ ID NO: 20 - DNA, 63 bp, Artificial Sequence
  Mellitin nucleotide sequence 1
atgaagtttcttgtgaatgtagcgctcgttttcatggtggtatatatctcatatatctatgcg

SEQ ID NO: 21 - DNA, 63 bp, Artificial Sequence
  Enterokinase cleavable linker
ggtggaggtggctctggaggcggcggctccggaggcggtggtagtgatgacgatgacaaagga

SEQ ID NO: 22 - DNA, 60 bp, Artificial Sequence
  GS-Linker
ggaggaggcggatcaggaggcggaggaagtggtggtggaggttctggtggtggcggtagt

SEQ ID NO: 23 - DNA, 30 bp, Artificial Sequence
  AVFAAASDAK
gctgtattcgctgcggcttccgatgccaaa

SEQ ID NO: 24 - DNA, 30 bp, Artificial Sequence
  AVFDRKSDAK
gctgtattcgataggaagtccgatgccaaa

SEQ ID NO: 25 - DNA, 27 bp, Artificial Sequence
  KILGRVFFV
aaaattttgggtagggttttttttgtg

SEQ ID NO: 26 - DNA, 27 bp, Artificial Sequence
  KLAEAIFKL
aagctcgcagaggcgattttcaaactg

SEQ ID NO: 27 - DNA, 27 bp, Artificial Sequence
  YAETAAFAY
tatgcagaaacggcggcatttgcttat

SEQ ID NO: 28 - DNA, 297 bp, Artificial Sequence
  Beta-2 microglobulin
atccaaagaactccgaagatccaagtgtactctagacaccctgctgaaaatggtaaatccaacttcctcaactgttacgtgagcggtttccacccgtctgatatagaggttgacctcctcaagaatggcgaacgcatcgaaaaggttgaacactcagacctctctttcagcaaagattggagcttttatcttttgtattacactgagtttaccccgacagagaaagacgaatatgcatgcagagtgaatcacgtgacgttgtcgcagccgaaaatcgtgaaatgggaccgcgatatg

SEQ ID NO: 29 - DNA, 819 bp, Artificial Sequence
  A*1101 Heavy chain
cattcgatgcgttacttctacacgtcggtgtctaggcccggcagaggagagccccgtttcatagcggtaggttacgttgacgacacgcaattcgtgcgttttgactcggacgccgcctcacaaagaatggaaccccgcgcgccctggattgagcaagagggacctgaatactgggatcaagaaacgaggaacgtgaaggcgcagtcgcaaactgatagggtcgatctgggaacccttaggggatattataaccaaagcgaagacggatctcatactattcaaattatgtatggttgcgacgttggccccgatggccgctttcttcgtggatacaggcaggacgcgtacgacggaaaggattatattgcgttgaatgaagatcttaggtcgtggacagcggcggacatggccgctcaaatcaccaaacgtaaatgggaagccgcacacgccgcagaacaacagcgcgcttatttggaaggcagatgtgtagagtggttgcgtcgttatctcgaaaacggtaaagaaacactccaacgcaccgatcccccgaagactcatatgactcaccacccaatatctgaccatgaggcgacgttgagatgttgggcattgggattctatccggcagaaataacgctcacatggcagcgtgacggagaggatcaaacgcaagatacggaacttgtggagacacgccctgcgggtgacggaacctttcagaagtgggcagcagttgtagtcccaagcggcgaggaacaacgctatacctgccacgtacaacacgaaggactccctaagccactcactctccgctgggaa

SEQ ID NO: 30 - DNA, 819 bp, Artificial Sequence
  A*0201 Heavy chain
catagtatgaggtatttctttacgagtgtgtcgcgccccggcagaggcgaacctcgctttatagctgtaggatatgtcgatgacacacaattcgtacgctttgattccgatgcagcgtctcaaagaatggagcctagagcgccgtggatagagcaagaaggccctgaatattgggatggcgagactcgcaaagtaaaggctcacagtcaaactcatcgtgtagatctgggcacgttgcgtggctattacaatcaatctgaagcaggatcgcacactgtacagcgtatgtacggctgcgatgttggcagcgactggcgctttcttagaggttaccatcaatatgcgtatgacggcaaggactacattgcattgaaggaggacttgaggagttggaccgcagcggatatggccgcgcagaccacaaagcataaatgggaggccgctcatgtcgctgaacagcttagagcttatttggaaggcacctgcgtagaatggctgcgccgttacttggagaacggcaaggaaaccttgcagaggaccgatgcgcctaaaacacatatgacacaccacgccgtatccgatcacgaagctaccctgaggtgttgggctctttccttttaccctgccgagattacattgacatggcagcgtgacggcgaagaccaaacacaagacactgagttggtggagacccgcccggctggcgacggaaccttccaaaagtgggccgctgtagtagtcccatccggacaggaacaacgctatacgtgtcacgttcaacatgaaggtctgcctaagccgctcaccttgcgctgggaa

SEQ ID NO: 31 - DNA, 819 bp, Artificial Sequence
  A*0101 Heavy chain
cattcaatgcgttacttctttacgtccgtatccaggccaggcaggggagagccgagattcatagctgtcggctacgtcgatgacacgcagttcgttcgtttcgatagcgacgccgcgtcccagaaaatggaacccagggctccgtggatcgagcaggagggaccggaatactgggatcaagagacacgtaatatgaaggcacacagtcaaacagatcgtgcaaacctcggaacgctgagaggttattataatcagtccgaagacggttcccacaccatacagattatgtacggatgtgacgtgggccctgatggcagattcctcagaggctaccgccaggacgcctatgatggcaaagattatattgccttgaatgaggatctgcgttcatggacggcggctgatatggccgcgcagatcacaaagagaaaatgggaagctgttcatgcggcggaacagcgccgtgtttacttggagggtaggtgcgtcgacggactgagacgttatttggagaatggcaaggaaacgcttcaaagaacggaccctcccaagacacacatgactcatcatcccatatctgatcatgaggcaactcttaggtgttgggctcttggcttctaccctgctgagattactcttacatggcaaagggacggcgaagatcagacccaagacactgagttggttgagaccagacctgcgggtgacggtacatttcagaaatgggcggcagtcgtggtgccgagcggtgaggaacaacgttacacatgtcatgttcagcatgaaggtctgcctaagccccttacactgcgctgggaa

SEQ ID NO: 32 - DNA, 45 bp, Artificial Sequence
  BirA recognition site
ctgcaccacatcttggacgcacaaaagatggtctggaatcaccgt

SEQ ID NO: 33 - DNA, 18 bp, Artificial Sequence
  His6X
catcaccatcatcaccac

SEQ ID NO: 34 - DNA, 1419 bp, Artificial Sequence
  A*1101-AVFDRKSDAK nucleotide
ggatccatgaagtttcttgtgaatgtagcgctcgttttcatggtggtatatatctcatatatctatgcggctgtattcgataggaagtccgatgccaaaggtggaggtggctctggaggcggcggctccggaggcggtggtagtgatgacgatgacaaaggaatccaaagaactccgaagatccaagtgtactctagacaccctgctgaaaatggtaaatccaacttcctcaactgttacgtgagcggtttccacccgtctgatatagaggttgacctcctcaagaatggcgaacgcatcgaaaaggttgaacactcagacctctctttcagcaaagattggagcttttatcttttgtattacactgagtttaccccgacagagaaagacgaatatgcatgcagagtgaatcacgtgacgttgtcgcagccgaaaatcgtgaaatgggaccgcgatatgggaggaggcggatcaggaggcggaggaagtggtggtggaggttctggtggtggcggtagtcattcgatgcgttacttctacacgtcggtgtctaggcccggcagaggagagccccgtttcatagcggtaggttacgttgacgacacgcaattcgtgcgttttgactcggacgccgcctcacaaagaatggaaccccgcgcgccctggattgagcaagagggacctgaatactgggatcaagaaacgaggaacgtgaaggcgcagtcgcaaactgatagggtcgatctgggaacccttaggggatattataaccaaagcgaagacggatctcatactattcaaattatgtatggttgcgacgttggccccgatggccgctttcttcgtggatacaggcaggacgcgtacgacggaaaggattatattgcgttgaatgaagatcttaggtcgtggacagcggcggacatggccgctcaaatcaccaaacgtaaatgggaagccgcacacgccgcagaacaacagcgcgcttatttggaaggcagatgtgtagagtggttgcgtcgttatctcgaaaacggtaaagaaacactccaacgcaccgatcccccgaagactcatatgactcaccacccaatatctgaccatgaggcgacgttgagatgttgggcattgggattctatccggcagaaataacgctcacatggcagcgtgacggagaggatcaaacgcaagatacggaacttgtggagacacgccctgcgggtgacggaacctttcagaagtgggcagcagttgtagtcccaagcggcgaggaacaacgctatacctgccacgtacaacacgaaggactccctaagccactcactctccgctgggaaccgggtagtctgcaccacatcttggacgcacaaaagatggtctggaatcaccgtcatcaccatcatcaccactagctgcag

SEQ ID NO: 35 - DNA, 1419 bp, Artificial Sequence
  A*1101-AVFAAASDAK nucleotide
ggatccatgaagtttcttgtgaatgtagcgctcgttttcatggtggtatatatctcatatatctatgcggctgtattcgctgcggcttccgatgccaaaggtggaggtggctctggaggcggcggctccggaggcggtggtagtgatgacgatgacaaaggaatccaaagaactccgaagatccaagtgtactctagacaccctgctgaaaatggtaaatccaacttcctcaactgttacgtgagcggtttccacccgtctgatatagaggttgacctcctcaagaatggcgaacgcatcgaaaaggttgaacactcagacctctctttcagcaaagattggagcttttatcttttgtattacactgagtttaccccgacagagaaagacgaatatgcatgcagagtgaatcacgtgacgttgtcgcagccgaaaatcgtgaaatgggaccgcgatatgggaggaggcggatcaggaggcggaggaagtggtggtggaggttctggtggtggcggtagtcattcgatgcgttacttctacacgtcggtgtctaggcccggcagaggagagccccgtttcatagcggtaggttacgttgacgacacgcaattcgtgcgttttgactcggacgccgcctcacaaagaatggaaccccgcgcgccctggattgagcaagagggacctgaatactgggatcaagaaacgaggaacgtgaaggcgcagtcgcaaactgatagggtcgatctgggaacccttaggggatattataaccaaagcgaagacggatctcatactattcaaattatgtatggttgcgacgttggccccgatggccgctttcttcgtggatacaggcaggacgcgtacgacggaaaggattatattgcgttgaatgaagatcttaggtcgtggacagcggcggacatggccgctcaaatcaccaaacgtaaatgggaagccgcacacgccgcagaacaacagcgcgcttatttggaaggcagatgtgtagagtggttgcgtcgttatctcgaaaacggtaaagaaacactccaacgcaccgatcccccgaagactcatatgactcaccacccaatatctgaccatgaggcgacgttgagatgttgggcattgggattctatccggcagaaataacgctcacatggcagcgtgacggagaggatcaaacgcaagatacggaacttgtggagacacgccctgcgggtgacggaacctttcagaagtgggcagcagttgtagtcccaagcggcgaggaacaacgctatacctgccacgtacaacacgaaggactccctaagccactcactctccgctgggaaccgggtagtctgcaccacatcttggacgcacaaaagatggtctggaatcaccgtcatcaccatcatcaccactagctgcag

SEQ ID NO: 36 - DNA, 1416 bp, Artificial Sequence
  A*0201-KILGRVFFV nucleotide
ggatccatgaaatttctcgttaatgttgcacttgtcttcatggtggtctatataagctacatatacgcgaaaattttgggtagggttttttttgtgggcggcggtggatctggcggaggcggttcaggcggaggcggtagtgatgacgatgataagggcatacagcgtactcctaagatccaggtttactctagacatccggctgagaacggcaaatcaaactttcttaattgctacgtcagcggctttcatccatcagatatagaggtcgacctgctgaagaatggcgaacgcattgaaaaagtcgaacatagtgacctctcattcagcaaagactggtccttttatctcctctactatacggagtttacacctactgaaaaggacgagtacgcctgtagagtaaatcatgtgacgctcagccagccgaagatagtgaaatgggatcgtgacatgggaggcggaggctctggaggcggtggctccggtggtggtggtagcggaggtggaggaagccatagtatgaggtatttctttacgagtgtgtcgcgccccggcagaggcgaacctcgctttatagctgtaggatatgtcgatgacacacaattcgtacgctttgattccgatgcagcgtctcaaagaatggagcctagagcgccgtggatagagcaagaaggccctgaatattgggatggcgagactcgcaaagtaaaggctcacagtcaaactcatcgtgtagatctgggcacgttgcgtggctattacaatcaatctgaagcaggatcgcacactgtacagcgtatgtacggctgcgatgttggcagcgactggcgctttcttagaggttaccatcaatatgcgtatgacggcaaggactacattgcattgaaggaggacttgaggagttggaccgcagcggatatggccgcgcagaccacaaagcataaatgggaggccgctcatgtcgctgaacagcttagagcttatttggaaggcacctgcgtagaatggctgcgccgttacttggagaacggcaaggaaaccttgcagaggaccgatgcgcctaaaacacatatgacacaccacgccgtatccgatcacgaagctaccctgaggtgttgggctctttccttttaccctgccgagattacattgacatggcagcgtgacggcgaagaccaaacacaagacactgagttggtggagacccgcccggctggcgacggaaccttccaaaagtgggccgctgtagtagtcccatccggacaggaacaacgctatacgtgtcacgttcaacatgaaggtctgcctaagccgctcaccttgcgctgggaaccaggctcattgcatcatattcttgatgcacagaaaatggtatggaatcatcgccatcatcatcatcatcactagctgcag

SEQ ID NO: 37 - DNA, 1416 bp, Artificial Sequence
  A*0201-KLAEAIFKL nucleotide
ggatccatgaagtttttggtgaacgtcgcgctcgtcttcatggtcgtttacatatcatacatttatgcgaagctcgcagaggcgattttcaaactgggaggaggtggttcgggcggaggcggcagtggaggtggtggctcagatgacgatgacaagggtattcaaaggacaccgaaaatccaagtgtattctagacatccggctgagaacggcaaatcaaactttcttaattgctacgtcagcggctttcatccatcagatatagaggtcgacctgctgaagaatggcgaacgcattgaaaaagtcgaacatagtgacctctcattcagcaaagactggtccttttatctcctctactatacggagtttacacctactgaaaaggacgagtacgcctgtagagtaaatcatgtgacgctcagccagccgaagatagtgaaatgggatcgtgacatgggaggcggaggctctggaggcggtggctccggtggtggtggtagcggaggtggaggaagccatagtatgaggtatttctttacgagtgtgtcgcgccccggcagaggcgaacctcgctttatagctgtaggatatgtcgatgacacacaattcgtacgctttgattccgatgcagcgtctcaaagaatggagcctagagcgccgtggatagagcaagaaggccctgaatattgggatggcgagactcgcaaagtaaaggctcacagtcaaactcatcgtgtagatctgggcacgttgcgtggctattacaatcaatctgaagcaggatcgcacactgtacagcgtatgtacggctgcgatgttggcagcgactggcgctttcttagaggttaccatcaatatgcgtatgacggcaaggactacattgcattgaaggaggacttgaggagttggaccgcagcggatatggccgcgcagaccacaaagcataaatgggaggccgctcatgtcgctgaacagcttagagcttatttggaaggcacctgcgtagaatggctgcgccgttacttggagaacggcaaggaaaccttgcagaggaccgatgcgcctaaaacacatatgacacaccacgccgtatccgatcacgaagctaccctgaggtgttgggctctttccttttaccctgccgagattacattgacatggcagcgtgacggcgaagaccaaacacaagacactgagttggtggagacccgcccggctggcgacggaaccttccaaaagtgggccgctgtagtagtcccatccggacaggaacaacgctatacgtgtcacgttcaacatgaaggtctgcctaagccgctcaccttgcgctgggaaccaggctcattgcatcatattcttgatgcacagaaaatggtatggaatcatcgccatcatcatcatcatcactagctgcag

SEQ ID NO: 38 - DNA, 1416 bp, Artificial Sequence
  A*0101- YAETAAFAY nucleotide
ggatccatgaaatttctcgttaatgttgcacttgtcttcatggtggtctatataagctacatatacgcgtatgcagaaacggcggcatttgcttatggcggcggtggatctggcggaggcggttcaggcggaggcggtagtgatgacgatgataagggcatacagcgtactcctaagatccaggtttactctagacatccggctgagaacggcaaatcaaactttcttaattgctacgtcagcggctttcatccatcagatatagaggtcgacctgctgaagaatggcgaacgcattgaaaaagtcgaacatagtgacctctcattcagcaaagactggtccttttatctcctctactatacggagtttacacctactgaaaaggacgagtacgcctgtagagtaaatcatgtgacgctcagccagccgaagatagtgaaatgggatcgtgacatgggaggcggaggctctggaggcggtggctccggtggtggtggtagcggaggtggaggaagccattcaatgcgttacttctttacgtccgtatccaggccaggcaggggagagccgagattcatagctgtcggctacgtcgatgacacgcagttcgttcgtttcgatagcgacgccgcgtcccagaaaatggaacccagggctccgtggatcgagcaggagggaccggaatactgggatcaagagacacgtaatatgaaggcacacagtcaaacagatcgtgcaaacctcggaacgctgagaggttattataatcagtccgaagacggttcccacaccatacagattatgtacggatgtgacgtgggccctgatggcagattcctcagaggctaccgccaggacgcctatgatggcaaagattatattgccttgaatgaggatctgcgttcatggacggcggctgatatggccgcgcagatcacaaagagaaaatgggaagctgttcatgcggcggaacagcgccgtgtttacttggagggtaggtgcgtcgacggactgagacgttatttggagaatggcaaggaaacgcttcaaagaacggaccctcccaagacacacatgactcatcatcccatatctgatcatgaggcaactcttaggtgttgggctcttggcttctaccctgctgagattactcttacatggcaaagggacggcgaagatcagacccaagacactgagttggttgagaccagacctgcgggtgacggtacatttcagaaatgggcggcagtcgtggtgccgagcggtgaggaacaacgttacacatgtcatgttcagcatgaaggtctgcctaagccccttacactgcgctgggaaccgggtagtctgcaccacatcttggacgcacaaaagatggtctggaatcaccgtcatcaccatcatcaccactagctgcag

SEQ ID NO: 39 - protein, 9 aa, Artificial Sequence
  EBV peptide 1
IVTDFSVIK

SEQ ID NO: 40 - protein, 9 aa, Artificial Sequence
  EBV peptide 2
NTLEQTVKK

SEQ ID NO: 41 - protein, 9 aa, Artificial Sequence
  CMV peptide
NLVPMVATV

SEQ ID NO: 42 - protein, 10 aa, Artificial Sequence
  CMV-derived peptide
VETEHDTLLY

SEQ ID NO: 43 - protein, 5 aa, Artificial Sequence
  Cleavable DDDDK peptide
DDDDK

SEQ ID NO: 44 - protein, 10 aa, Artificial Sequence
  GTSGSPIVNR
GTSGSPIVNR

SEQ ID NO: 45 - protein, 9 aa, Artificial Sequence
  ATYGWNLVK
ATYGWNLVK

SEQ ID NO: 46 - protein, 9 aa, Artificial Sequence
  SIIPSGPLK
SIIPSGPLK

SEQ ID NO: 47 - protein, 10 aa, Artificial Sequence
  RMVLASTTAK
RMVLASTTAK

SEQ ID NO: 48 - protein, 9 aa, Artificial Sequence
  KSMREEYRK
KSMREEYRK

SEQ ID NO: 49 - protein, 9 aa, Artificial Sequence
  ATIGTAMYK
ATIGTAMYK

SEQ ID NO: 50 - protein, 10 aa, Artificial Sequence
  SSCSSCPLSK
SSCSSCPLSK

SEQ ID NO: 51 - protein, 9 aa, Artificial Sequence
  YVNVNMGLK
YVNVNMGLK

SEQ ID NO: 52 - protein, 9 aa, Artificial Sequence
  FLDKGTYTL
FLDKGTYTL

SEQ ID NO: 53 - protein, 9 aa, Artificial Sequence
  VLEETSVML
VLEETSVML

SEQ ID NO: 54 - protein, 9 aa, Artificial Sequence
  FLYALALLL
FLYALALLL